(19) United States
(12) Patent Application Publication
(10) Pub. No.: US 2014/0107568 A1
(43) Pub. Date: Apr. 17, 2014

(54) METHOD OF DETERMINING AN INJECTED DOSE BASED ON A KNOWN ORAL DOSE

(71) Applicant: ANTARES PHARMA, INC., Ewing, NJ (US)

(72) Inventors: Peter L. Sadowski, Woodbury, MN (US); Kaushik J. Dave, Edison, NJ (US); Paul K. Wotton, Newtown, PA (US); Hermanus LeRoux Jooste, Villanova, PA (US)

(21) Appl. No.: 13/676,257

(22) Filed: Nov. 14, 2012

Related U.S. Application Data
(60) Provisional application No. 61/713,197, filed on Oct. 12, 2012.

(51) Int. Cl. A61K 31/519; A61M 5/30

(52) U.S. Cl. CPC A61K 31/519; A61M 5/30
USPC 604/68; 544/256; 514/249

(57) ABSTRACT

The present disclosure relates to methods of determining dosages of compositions used in injection devices, in view of known oral doses of the same compositions.

### CROSS-REFERENCE TO RELATED APPLICATIONS

This application claims priority to U.S. Provisional Patent Application No. 61/713,197, filed on Oct. 12, 2012, which is incorporated by reference herein in its entirety.

### FIELD OF THE DISCLOSURE

The present disclosure relates to methods of determining dosages of compositions used in injection devices, in view of known oral doses of the same compositions.

### BACKGROUND INFORMATION

Hazardous agents, such as cytotoxic agents, have been useful in managing and treating a number of diseases such as rheumatoid arthritis (and other autoimmune diseases), juvenile rheumatoid arthritis, psoriatic arthritis, systemic lupus erythematosus, steroid-resistant polymyositis or dermatomyositis, Wegener's granulomatosis, polyarteritis nodosa, and some forms of vasculitis. Hazardous agents tend to exhibit side effects, however, that are harmful or toxic to the subject. Many of these side effects occur when hazardous agents are administered orally, but the oral form is generally the preferred method of delivery of these agents due to its ease of use.

In addition to increased toxicity, variable and reduced bioavailability has been observed for some hazardous agents, such as methotrexate, that are orally administered. These limitations are particularly demonstrated when the oral dosing is escalated beyond 15 mg per dose. It has been suggested that with parenteral administration, such as by injection, more predictable, reproducible and complete bioavailability along with better therapeutic results could be achieved, particularly at higher dosages.

Because of the large number of precautions that must be learned and followed in order to safely inject a hazardous agent, and to inject a hazardous agent in therapeutically accurate amounts, it is presently thought that it is not practical for hazardous agents, and particularly methotrexate, to be self-injected by a patient outside of a clinical setting or without the assistance of a health care provider.

### SUMMARY

In an embodiment, disclosed herein is a method of administering a medicament to a subject subcutaneously, comprising identifying a suitable oral dose of a medicament, converting the oral dose of the medicament to an equivalent subcutaneous dose of the medicament by multiplying the oral dose of the medicament by 0.6101 and adding 2.9274 to the product, thereby converting the oral dose to an equivalent subcutaneous dose, and administering the equivalent subcutaneous dose of the medicament to the subject subcutaneously.

In an embodiment, also disclosed herein is a method of determining the amount of a medicament to administer to a subject subcutaneously, comprising identifying a suitable oral dose of a medicament, and converting the oral dose of the medicament to an equivalent subcutaneous dose of the medicament by multiplying the oral dose of the medicament by 0.6101 and adding 2.9274 to the product, thereby determining the amount of the medicament to administer to the subject subcutaneously.

In an embodiment, also disclosed is a method of converting a preselected oral dose of a medicament to an equivalent subcutaneous dose of the medicament, comprising multiplying the oral dose of the medicament by 0.6101 and adding 2.9274 to the product, thereby converting the oral dose to an equivalent subcutaneous dose.

In an embodiment, also disclosed herein is a method of converting an oral dose of a medicament to an equivalent subcutaneous dose of the medicament, comprising identifying a suitable oral dose of the medicament for a subject, multiplying the oral dose of the medicament by 0.6101 and adding 2.9274 to the product, thereby converting the oral dose to an equivalent subcutaneous dose, wherein a suitable oral dose of the medicament is a therapeutically effective amount of the medicament.

In some embodiments, a subcutaneous dose is a dose that is delivered to a subject by way of a jet injector device. In some embodiments, a jet injector device is a needle-assisted jet injector.

In some embodiments, a kit is provided for administration of a medicament to a subject in need thereof, comprising a jet injector device for subcutaneous administration of the medicament to the subject and instructions for converting an oral dose of the medicament to an equivalent subcutaneous dose of the medicament for use in conjunction with the jet injector. Moreover, it is important for a patient or a healthcare provider to obtain the proper dose.

In an embodiment, the medicament comprises methotrexate.

In an embodiment, disclosed herein is a method of administering a medicament comprising methotrexate to a subject subcutaneously via a needle-assisted jet injector, comprising identifying a suitable oral dose of the medicament comprising methotrexate, converting the oral dose of the medicament comprising methotrexate to an equivalent subcutaneous dose of the medicament by multiplying the oral dose of the medicament by 0.6101 and adding 2.9274 to the product, thereby converting the oral dose to an equivalent subcutaneous dose, and administering the equivalent subcutaneous dose of the medicament comprising methotrexate to the subject subcutaneously via a needle-assisted jet injector.

In an embodiment, also disclosed herein is a method of determining the amount of a medicament comprising methotrexate to administer to a subject subcutaneously via a needle-assisted jet injector, comprising identifying a suitable oral dose of a medicament comprising methotrexate, and converting the oral dose of the medicament comprising methotrexate to an equivalent subcutaneous dose of the medicament comprising methotrexate by multiplying the oral dose of the medicament comprising methotrexate by 0.6101 and adding 2.9274 to the product, thereby determining the amount of the medicament comprising methotrexate to administer to the subject subcutaneously via a needle-assisted jet injector.

In an embodiment, encompassed herein is a method of determining an administrable dose of a medicament comprising methotrexate, wherein the administrable dose is administrable via needle-assisted jet injector, the method comprising identifying a suitable oral dose of a medicament comprising methotrexate, converting the oral dose of the medicament comprising methotrexate to an equivalent subcutaneous dose of the medicament comprising methotrexate by multiplying the oral dose of the medicament comprising methotrexate by 0.6101 and adding 2.9274 to the product, and converting the equivalent subcutaneous dose to a commercially-available administrable dose. In an embodiment, the method of determining an administrable dose comprises comparing the bioavailability of the corresponding subcutaneous dose with the bioavailability of the administrable dose.

### BRIEF DESCRIPTION OF THE DRAWINGS

These and other objects, features and advantages of the invention will be apparent from a consideration of the following non-limiting detailed description considered in conjunction with the drawing figures, in which:

FIG. 1 is a graph illustrating an equation developed to determine the correlation between an oral dose and an equivalent jet-injected dose of a medicament.

### DETAILED DESCRIPTION

Various embodiments of the present invention are described more fully below. Some but not all embodiments of the present invention are shown. Indeed, various embodiments of the invention may be embodied in many different forms and should not be construed as limited to the embodiments expressly described. Like numbers refer to like elements throughout. The singular forms “a,” “an,” and “the” include the singular and plural unless the context clearly dictates otherwise.

Methods

In an embodiment, to improve, among other things, the accuracy, efficacy, safety, and efficiency of dosage of therapeutic agents, the correlation between oral and equivalent injectable dosage must be understood. The present disclosure addresses this need.

In an embodiment, disclosed herein is a method of administering a medicament to a subject, comprising identifying a suitable oral dose of a medicament, converting the oral dose of the medicament to an equivalent peritoneal (e.g., subcutaneous, intravenous, intramuscular, or intradermal) dose of the medicament, and administering the equivalent dose of the medicament to the subject. In an embodiment, an equivalent peritoneal dose of the medicament is further converted into an administrable dose. In an embodiment, an administrable dose is based on the commercially available dose of the medicament that is closest in value (e.g., mg/ml) to the equivalent dose of the medicament.

In one embodiment, the oral dose is converted to an equivalent subcutaneous dose by providing an algorithm and applying the algorithm to the oral dose. Optionally, the algorithm can be provided on a computer-readable medium. In an embodiment, the corresponding subcutaneous dose is obtained by multiplying the oral dose of the medicament by about 0.6101 and adding about 2.9274 to the product, thereby converting the oral dose to an equivalent subcutaneous dose. In an embodiment, more simply, the corresponding subcutaneous dose is obtained by multiplying the oral dose of the medicament by about 0.6 and adding about 3 to the product, thereby converting the oral dose to an equivalent subcutaneous dose. In an embodiment, a method further comprises the step of obtaining an administrable dose based on the equivalent subcutaneous dose. In an embodiment, an administrable dose is based on one or more commercially available doses. In an embodiment, an administrable dose is based on the commercially-available dose that is closest to the equivalent subcutaneous dose.

In an embodiment, the subcutaneous dose is calculated for delivery by a jet injector. In an embodiment, the subcutaneous dose is calculated for delivery by a needle-assisted jet injector. Such injectors are described in greater detail elsewhere herein.

In one embodiment, a method is provided for determining the amount of a medicament to administer to a subject subcutaneously, comprising identifying a suitable oral dose of a medicament, then converting the oral dose of the medicament to an equivalent subcutaneous dose of the medicament by applying an algorithm designed to correlate the oral dose of a medicament to a corresponding equivalent subcutaneous dose of the medicament. In an embodiment, an equivalent subcutaneous dose of a medicament is a dose of the medicament that, when administered subcutaneously to a subject, results in bioequivalence with the orally administered dose. In an embodiment, an equivalent subcutaneous dose of a medicament is a dose of the medicament that, when administered subcutaneously to a subject, is therapeutically equivalent to the orally administered dose. In an embodiment, an equivalent subcutaneous dose of a medicament is a dose of the medicament that, when administered subcutaneously to a subject, is substantially bioequivalent to the orally administered dose. In an embodiment, the medicament comprises methotrexate.

In some embodiments, an equivalent dose is calculated based on bioavailability of the therapeutic agent. Such methods can be found in U.S. Patent Application Publication 2012/0157965, U.S. patent application Ser. No. 13/564,693, and PCT/US2012/049235, each of which is incorporated herein by reference in its entirety.

In some embodiments, an equivalent dose of a therapeutic agent is determined by assessing the bioavailability of the therapeutic agent. In some embodiments, the bioavailability of a therapeutic agent can be approximated to a known or desired level by selecting one or more factors in the configuration of a jet injector, to maintain bioequivalence for the therapeutic agent. In an embodiment, bioequivalence can be measured using means known in the art to measure plasma levels to determine the rate and extent of absorption of the therapeutic agent and determining the extent of absorption. One such measure known in the art is the determination of area under the concentration curve, the AUC, which is directly proportional to the amount of therapeutic agent in the patients's blood. Another such measure is the determination of the peak serum concentration of therapeutic agent, the C. In an embodiment, bioequivalence may be established if a therapeutic agent injected via an injector according to the present disclosure reaches the site of absorption in similar times and is absorbed to the same extent as if the hazardous agent had been introduced to the subject via other known routes of administration. In some embodiments, an equivalent dose of a therapeutic agent is determined by a method comprising assessing the indirect actions of the therapeutic agent after administration.

In some embodiments, also disclosed herein are methods of converting an oral dose of a medicament to an equivalent subcutaneous dose of the medicament, comprising identifying a suitable oral dose of the medicament for a subject, providing an algorithm to convert the oral dose to an equivalent subcutaneous dose, and applying the algorithm to the oral dose, thereby converting the oral dose to an equivalent subcutaneous dose, wherein a suitable oral dose of the medicament is a therapeutically effective amount of the medicament. In some embodiments, the methods include a determination of bioavailability, as described in detail elsewhere herein, based on a specific medicament and/or route of administration. In some embodiments, the medicament comprises methotrexate.

In some embodiments, also disclosed herein are methods of converting an oral dose of a medicament to an equivalent subcutaneous dose of the medicament, in view of a specific injector to be used for administration of the subcutaneous dose, comprising identifying a suitable oral dose of the medicament for a subject, identifying a suitable injector device for administering the subcutaneous dose of the medicament, providing an algorithm to convert the oral dose to an equivalent subcutaneous dose to be delivered by the injector device, and applying the algorithm to the oral dose, thereby converting the oral dose to an equivalent subcutaneous dose, wherein a suitable oral dose of the medicament is a therapeutically effective amount of the medicament. Such injectors are described in and encompassed by U.S. Patent Application Publication 2012/0157965, U.S. patent application Ser. No. 13/564,693, International application PCT/US2012/049235, and U.S. Pat. Nos. 6,746,429, 7,744,582, 7,776,015, and 8,021,335, each of which is incorporated herein by reference in its entirety. In some embodiments, the methods include a determination of therapeutic equivalence, as described in detail elsewhere herein, based on a specific injector device. In some embodiments, the methods include a determination of bioavailability, as described in detail elsewhere herein, based on a specific injector device. In some embodiments, the medicament comprises methotrexate and the injector device comprises a needle-assisted injector device.

Therapeutic Agents

In an embodiment, a therapeutic agent, also referred to herein as a medicament, is methotrexate. Other therapeutic agents include, but are not limited to, rifampin, sulfonamides, chloramphenicol, fluoroquinolones, aminopenicilin, natural penicillin, ampicillin, cloxacilin, isoxicam, diclofenac, ketoprofen, acetylsalicylate, meloxicam, tenoxicam, vitamin B6, vitamin B12, and vitamin K, and combinations thereof.

In an embodiment, a dose of a therapeutic agent is identified as a suitable dose. In an embodiment, a suitable dose of a therapeutic agent is an amount of therapeutic agent understood and/or believed to be an amount sufficient to bring about a desired effect in a subject intended to receive the therapeutic agent. In an embodiment, a suitable dose of a therapeutic agent is a therapeutically effective amount of the therapeutic agent. It will be understood by the skilled artisan, when armed with the disclosure encompassed herein, that additional factors may need to be considered for a selected therapeutic agent, including, but not limited to, whether there are any active or passive transport mechanisms that must be taken into account, whether the locus of injection affects the rate and/or degree of uptake of the therapeutic agent, whether injection of the therapeutic agent can affect the activity or availability of the therapeutic agent, and whether the concentration or form of the therapeutic agent can affect the uptake and/or bioavailability of therapeutic agent differently via injection than via oral administration.

In one embodiment, a therapeutic agent is provided in a therapeutically effective amount. In an aspect, an oral dose that forms the basis for the determination of an equivalent subcutaneous dose is a dose in a therapeutically effective amount. In an embodiment, a therapeutically effective amount of a therapeutic agent is a suitable dose. “Therapeutically effective amount” refers to the amount of a therapeutic agent that, when administered to a subject for treating a disease or disorder, or at least one of the clinical symptoms of a disease or disorder, is sufficient to affect such treatment of the disease, disorder, or symptom. The therapeutically effective amount may vary depending, for example, on the compound, the disease, disorder, and/or symptoms of the disease, severity of the disease or disorder, and/or symptoms of the disease or disorder, the age, weight, and/or health of the patient to be treated, and the judgment of the prescribing physician. A therapeutically effective amount may be ascertained by those skilled in the art or capable of determination by routine experimentation. In an embodiment, a therapeutically effective amount is determined on the same clinical outcome being defined by an oral dose and by a dose administered via an alternate route of administration (e.g., subcutaneous).

In some embodiments, the therapeutic agent is methotrexate and/or one or more derivatives of methotrexate. Methotrexate and derivatives of methotrexate, as well as the pharmacokinetics of methotrexate, administered both orally an injected, are described in greater detail in U.S. Patent Application Publication 2012/0157965, incorporated herein by reference in its entirety. In some embodiments, encompassed are methotrexate and/or derivatives of methotrexate and/or pharmaceutically acceptable salts, solvates, hydrates, oxides and N-oxides thereof, are injected. In some embodiments, the present disclosure relates to the methotrexate and/or derivatives of methotrexate and one or more pharmaceutically acceptable excipients. In some embodiments, the present disclosure relates to a pharmaceutically acceptable salt of methotrexate and/or derivatives of methotrexate. In some embodiments, the present disclosure relates to pharmaceutical compositions comprising methotrexate and a pharmaceutically acceptable excipient.

Injection

In some embodiments, a parenteral dose is a dose that is delivered to a subject by way of an injector device. In some embodiments, a subcutaneous, intravenous, intramuscular, or intradermal dose is a dose that is delivered to a subject by way of an injector device. In some embodiments, a subcutaneous dose is a dose that is delivered to a subject by way of a jet injector device. In some embodiments, a jet injector device does not comprise a needle. In some embodiments, a jet injector device does not comprise the use of a needle. In some embodiments, a jet injector device is a needle-assisted jet injector, also referred to as a high pressure auto-injector. In some embodiments, a jet injector device is a needle assisted jet injector comprising a syringe.

In some embodiments, the present disclosure encompasses the conversion of a parenterally administered medicament dose to an equivalent dose of the therapeutic agent for subcutaneous injection via a jet injector. In some embodiments, the jet injector is a needle-assisted jet injector.

In some embodiments, the present disclosure relates to the conversion of an oral dose of a therapeutic agent to an equivalent dose of the therapeutic agent for subcutaneous injection via a jet injector. In some embodiments, the jet injector is a needle-assisted jet injector. In some embodiments, the jet injector is a needle-free jet injector.

Non-limiting examples of suitable injectors and injection methods can be found in U.S. Patent Application Publication 2012/0157965, U.S. patent application Ser. No. 13/564,693, International application PCT/US2012/049235, and U.S. Pat. Nos. 6,746,429, 7,744,582, 7,776,015, and 8,021,335, each of which is incorporated herein by reference in its entirety. As will be understood by the skilled artisan, when armed with the disclosure encompassed herein, one or multiple factors may be used to alter the delivery of a medicament via an injector, and the subsequent bioavailability and/or therapeutic effect, including, but not limited to, the size of the injected dose, the formulation of the injected dose, the speed of the injection, the method of injection, the use of a needle to assist injection, the depth of the injector, the depth of the needle, and the site of injection, among other factors.

In an embodiment, a powered injector may be used. In an embodiment, an injector encompassed by the present disclosure uses an energy source that produces moderate to high pressure in the medicament chamber so that a medicament contained in the medicament chamber is fired at a fast speed and is completely injected into a subject in less than about 5 seconds. In another embodiment, the powered injector completely delivers the dose into a subject at a rate that is at least about twice as fast as the comparator injector or delivery system. Other embodiments of the powered injectors are jet injectors, which can be needle-assisted or needle-free jet injectors. Jet injector embodiments can be configured to have an energy source selected to produce a high pressure in the medicament chamber to eject the medicament with sufficient pressure, force, and speed to exit the injector as a fluid jet.

In some embodiments, a kit is provided for administration of a medicament to a subject in need thereof, comprising a jet injector device as encompassed herein for administration (e.g., subcutaneous) of the medicament to the subject and instructions for converting an oral dose of the medicament to an equivalent subcutaneous, intravenous, intradermal, or intramuscular dose of the medicament, as described and encompassed herein, for use in conjunction with the jet injector.

### EXPERIMENTAL EXAMPLES

An algorithm was developed to correlate a known oral dose of methotrexate to a jet injector dose. Table 1 illustrates the calculated correlation between oral doses and needle-assisted jet injector doses of methotrexate. The second column illustrates the precise subcutaneous dose calculated based on the algorithm, described in detail below. The third column represents the administrable dose, based on commercially available doses, closest in value to the precise calculated dose.

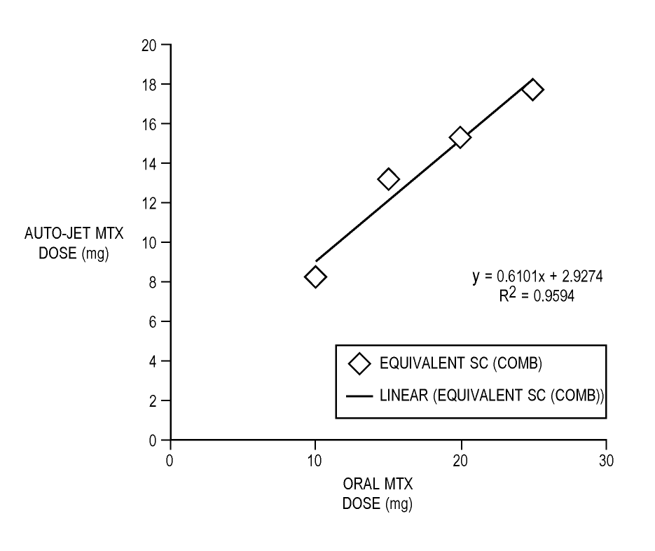

Based on the data set forth in table 1, a line equation of y=mx+b was developed to correlate an oral dose of methotrexate to an equivalent dose for use with a jet injector. In the present equation, illustrated by the graph shown in FIG. 1, the slope, m, was determined to be 0.6101 and a y-intercept, b, to be 2.9274. The correlation coefficient of the line, R², is 0.9594. As shown in FIG. 1, the x value represents the known oral dose of methotrexate, and the y value, which can be obtained using the algorithm described herein, will provide the corresponding jet injector dose.

TABLE 1
Oral to jet injector dose conversion for methotrexate.

| Oral Dose | Precise Subcutaneous Dose | Selected Administrable |
|---|---|---|
| (mg) | (mg) | Dose (mg) |
| 7.5 | 7.50315 | 7.5 |
| 10 | 9.0284 | 10 |
| 12.5 | 10.55365 | 10 |
| 15 | 12.0789 | 10 |
| 17.5 | 13.60415 | 15 |
| 20 | 15.1294 | 15 |
| 22.5 | 16.65465 | 15 |
| 25 | 18.1799 | 20 |
| 27.5 | 19.70515 | 20 |
| 30 | 21.2304 | 20 |

Each and every reference herein is incorporated by reference in its entirety. The term “about,” as used herein, should generally be understood to refer to both the corresponding number and a range of numbers. Moreover, all numerical ranges herein should be understood to include each whole integer within the range.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

1. A method of administering methotrexate to a subject subcutaneously, the method comprising:
(a) identifying a suitable oral dose of methotrexate for a subject;
(b) calculating an equivalent subcutaneous dose by multiplying the oral dose of methotrexate by 0.6101 and adding 2.9274 to the product, thereby converting the oral dose of methotrexate to the equivalent subcutaneous dose; and
(c) administering the equivalent subcutaneous dose of methotrexate to the subject subcutaneously,
wherein the suitable oral dose of methotrexate is a therapeutically effective amount of methotrexate and is between 7.5 mg and 30 mg.

2. (canceled)

3. (canceled)

4. (canceled)

5. The method of claim 1, wherein the subcutaneous dose is a dose that is delivered to a subject by way of a jet injector device.

6. The method of claim 5, wherein the jet injector device is a needle-assisted jet injector.

7. (canceled)

8. The method of claim 1, wherein the oral dose and the subcutaneous dose are selected from the group consisting of an oral dose of 7.5 mg and an equivalent subcutaneous dose of about 7.50315 mg, an oral dose of 10 mg and an equivalent subcutaneous dose of about 9.0284 mg, an oral dose of 12.5 mg and an equivalent subcutaneous dose of about 10.55365 mg, an oral dose of 15 mg and an equivalent subcutaneous dose of about 12.0789 mg, an oral dose of 17.5 mg and an equivalent subcutaneous dose of about 13.60415 mg, an oral dose of 20 mg and an equivalent subcutaneous dose of about 15.1294 mg, an oral dose of 22.5 mg and an equivalent subcutaneous dose of about 16.65465 mg, an oral dose of 25 mg and an equivalent subcutaneous dose of about 18.1799 mg, an oral dose of 27.5 mg and an equivalent subcutaneous dose of about 19.70515 mg, and an oral dose of 30 mg and an equivalent subcutaneous dose of about 21.2304 mg.

9. The method of claim 1, wherein the subcutaneous dose is an administrable dose, further wherein the oral dose and the administrable dose are selected from the group consisting of an oral dose of 7.5 mg and an administrable dose of 7.5 mg, an oral dose of 10 mg and an administrable dose of 10 mg, an oral dose of 12.5 mg and an administrable dose of 10 mg, an oral dose of 15 mg and an administrable dose of 10 mg, an oral dose of 17.5 mg and an administrable dose of 15 mg, an oral dose of 20 mg and an administrable dose of 15 mg, an oral dose of 22.5 mg and an administrable dose of 15 mg, an oral dose of 25 mg and an administrable dose of 20 mg, an oral dose of 27.5 mg and an administrable dose of 20 mg, and an oral dose of 30 mg and an administrable dose of 20 mg.

10. A kit for administration of methotrexate to a subject in need thereof, the kit comprising:
(a) a jet injector device for subcutaneous administration of the medicament to the subject; and
(b) instructions for converting an oral dose of methotrexate to an equivalent subcutaneous dose of methotrexate for use in conjunction with the jet injector,
wherein the instructions describe the method of claim 1.

11. (canceled)

12. (canceled)

13. (canceled)

14. The kit of claim 10, wherein the oral dose and the subcutaneous dose are selected from the group consisting of an oral dose of 7.5 mg and an equivalent subcutaneous dose of about 7.50315 mg, an oral dose of 10 mg and an equivalent subcutaneous dose of about 9.0284 mg, an oral dose of 12.5 mg and an equivalent subcutaneous dose of about 10.55365 mg, an oral dose of 15 mg and an equivalent subcutaneous dose of about 12.0789 mg, an oral dose of 17.5 mg and an equivalent subcutaneous dose of about 13.60415 mg, an oral dose of 20 mg and an equivalent subcutaneous dose of about 15.1294 mg, an oral dose of 22.5 mg and an equivalent subcutaneous dose of about 16.65465 mg, an oral dose of 25 mg and an equivalent subcutaneous dose of about 18.1799 mg, an oral dose of 27.5 mg and an equivalent subcutaneous dose of about 19.70515 mg, and an oral dose of 30 mg and an equivalent subcutaneous dose of about 21.2304 mg.

15. The kit of claim 10, wherein the subcutaneous dose is an administrable dose, further wherein the oral dose and the administrable dose are selected from the group consisting of an oral dose of 7.5 mg and an administrable dose of 7.5 mg, an oral dose of 10 mg and an administrable dose of 10 mg, an oral dose of 12.5 mg and an administrable dose of 10 mg, an oral dose of 15 mg and an administrable dose of 10 mg, an oral dose of 17.5 mg and an administrable dose of 15 mg, an oral dose of 20 mg and an administrable dose of 15 mg, an oral dose of 22.5 mg and an administrable dose of 15 mg, an oral dose of 25 mg and an administrable dose of 20 mg, an oral dose of 27.5 mg and an administrable dose of 20 mg, and an oral dose of 30 mg and an administrable dose of 20 mg.

16. A method of administering methotrexate to a subject subcutaneously, the method comprising:
(a) identifying a suitable oral dose of methotrexate for a subject;
(b) calculating an equivalent subcutaneous dose by multiplying the oral dose of methotrexate by about 0.6 and adding about 3 to the product;
(c) converting the oral dose of methotrexate to the equivalent subcutaneous dose; and
(d) administering the equivalent subcutaneous dose of methotrexate to the subject subcutaneously,
wherein the suitable oral dose of methotrexate is a therapeutically effective amount of methotrexate and is greater than or equal to 7.5 mg.
